(19) United States
(12) Patent Application Publication
(10) Pub. No.: US 2012/0220807 A1
(43) Pub. Date: Aug. 30, 2012

(54) a-OLEFIN / VINYL PYRROLIDINONE COPOLYMERS AS ASPHALTENE DISPERSANTS

(75) Inventor: Richard F. Miller, Humble, TX (US)

(73) Assignee: SHRIEVE CHEMICAL PRODUCTS, INC., The Woodlands, TX (US)

(21) Appl. No.: 13/220,478

(22) Filed: Aug. 29, 2011

Related U.S. Application Data
(60) Provisional application No. 61/385,753, filed on Sep. 23, 2010.

(51) Int. Cl. C07C 7/20

(52) U.S. Cl. 585/4

(57) ABSTRACT

An additive comprising, dissolved in a solvent, an asphaltene dispersant selected from the group consisting of α-olefin/vinyl pyrrolidinone copolymers. The copolymer can comprise at least one α-olefin selected from the group consisting of mono-α-olefins, at least one α-olefin selected from the group consisting of linear α-olefins and/or at least one α-olefin is selected from the group consisting of 1-hexadecene, 1-octadecene, 1-eicosene, 1-docosene, 1-tetracosene, 1-hexacosene, 1-octacosene and 1-triacontene. The additive can comprise a copolymer of 2-pyrrolidinone, 1-ethenyl monomer. The copolymer can have a Hansen Solubility Parameter (HSP), δ, of greater than 16, 17 or 18 MPa^{1/2}. The solvent can be selected from the group consisting of aromatic solvents, such as 1-methyl naphthalene, bis-(m-phenoxyphenyl)ether, o-xylene, toluene and heavy aromatic solvents. Also provided is a method of inhibiting asphaltene precipitation in a fluid by introducing into the fluid an asphaltene dispersant selected from the group consisting of α-olefin/vinyl pyrrolidinone copolymers.

### CROSS-REFERENCE TO RELATED APPLICATIONS

This application claims the benefit under 35 U.S.C. §119(e) of U.S. Provisional Patent Application No. 61/385,753 filed Sep. 23, 2010, the disclosure of which is hereby incorporated herein by reference.

### STATEMENT REGARDING FEDERALLY SPONSORED RESEARCH OR DEVELOPMENT

Not applicable.

### BACKGROUND

1. Field of the Invention

This invention relates generally to petroleum recovery and refining. More specifically, this invention relates to prevention and/or minimization of asphaltene precipitation. Still more specifically, this invention relates to asphaltene dispersants suitable for inhibiting asphaltene precipitation.

2. Background of Invention

Precipitation of asphaltenes, for example in reservoirs, wells and distribution and refining facilities, can have a substantial detrimental effect on the economics of oil production. Such asphaltene precipitation can result in a reduction in well productivity and can also lead to plugging of piping in distribution and refining facilities.

The behavior of asphaltenes in crude oils is complex. Asphaltenes are heterocyclic macromolecules consisting primarily of carbon, hydrogen, and lesser amounts of components such as, but not limited to, sulfur, nitrogen and oxygen. Resins and maltenes have structures similar to asphaltenes, with significantly lower molecular weights. It is generally accepted that resins and maltenes are responsible for maintaining dispersion of asphaltene particles. It is theorized that the asphaltenes are surrounded by the polar heads of the resins and/or maltenes while the nonpolar alkyl tails thereof interact with the oil phase. Therefore, crude oils having high ratio of resins and/or maltenes to asphaltenes are less likely to exhibit asphaltene deposition than crudes comprising large amounts of nonpolar saturates relative to aromatics. The latter crude oils have a higher propensity to exhibit asphaltene-precipitation problems.

At ‘normal’ operating (e.g. reservoir) conditions, thermodynamic equilibrium is maintained among the asphaltenes, resins and maltenes in the oil phase. This equilibrium of the system can be perturbed by a plethora of factors, including, without limitation, decline of the (e.g. reservoir) pressure toward the bubblepoint, change in the temperature, and the addition to the oil of one or more miscible solvents that are not compatible with the asphaltene molecule(s). Such miscible solvents are frequently introduced, for example, during various Enhanced Oil Recovery (EOR) operations.

Substantial effort has been devoted by the oil industry to the development of practical and effective solutions to the problem of asphaltene deposition in reservoirs, wells, distribution and refining equipment/facilities. To date, a variety of approaches to addressing the problem of asphaltene deposition have been investigated, each approach having certain advantages and concomitant drawbacks. One approach involves the physical removal of deposits through wireline bailing, drilling, hydroblasting and/or scraping. Ultrasonic treatment techniques have also been proposed to address asphaltene deposition by ultrasonically breaking up the asphaltene aggregate and thereby reducing its viscosity. The most widely used approaches to asphaltene control involve soaks with aromatic solvent(s) and/or treatment with polymeric dispersants that inhibit asphaltene flocculation.

Two popular competing asphaltene molecular models differ in the predicted distribution of the fused aromatic rings and aliphatic moieties. In the ‘continental’ model, asphaltenes are composed of a large poly-condensed aromatic center with an aliphatic periphery. Conversely, in the ‘archipelago’ model, asphaltenes are depicted as smaller poly-condensed aromatic ‘islands’ that are interconnected via aliphatic chains.

Results from recent structural analyses add to the growing support of the ‘archipelago’ asphaltene structure. For example, Strausz, O. P., Peng, P, and Murgich, J., Energy Fuels, 2002, 16(4), 809, used gel permeation chromatography to demonstrate the long-time dissociation (on the scale of days to weeks) of large asphaltene aggregates. This long-time dissociation is presumably due to the large number of intermolecular neighbors (and thus interactions) possible among archipelago-type structures. A study of asphaltene pyrolysis residues also revealed that the saturate, aromatic and polar-aromatic composition of such residues was most consistent with the aliphatic-bridged fused aromatic ring, or archipelago model.

Additionally, recent small-angle neutron scattering (SANS) characterization of asphaltene aggregates quantified a significant degree of solvent entrainment by said aggregates (0.4-0.6% v/v). Such entrainment is presumably within cavities that are plausible only in archipelago-type asphaltenic aggregates. This work is a first regarding these levels of asphaltenic aggregate description by an in situ characterization technique like SANS.

Interactions within asphaltenic aggregates are largely due to van der Waals (dispersion) forces and include π-π overlap between the aromatic-rich regions of the asphaltenes with possible enhancement of association by coulombic interactions (hydrogen-bonding) among polar oxygenic and nitrogenous moieties.

Assuming the molecular structure of the asphaltene aggregate fits the ‘archipelago’ model, it follows that an effective dispersant should have a similar structure, i.e. condensed aromatic-like ‘islands’ interconnected with aliphatic chains. Further, the dispersant and the solvent used to deliver the dispersant should have similar thermodynamic properties to those of the asphaltene aggregate.

Solubility parameters have found their greatest use in the coatings industry to aid in the selection of solvents. They are also used in other industries, however, to predict compatibility of polymers, permeation rates and even to characterize the surfaces of various pigments, fibers and fillers. Liquids with similar solubility parameters (δ) are miscible. Likewise, polymers will dissolve in solvents/liquids whose solubility parameters are similar to the solubility parameter of the polymer.

Generally, dispersants employed as asphaltene precipitation inhibitors are polymers believed to have characteristics in common with petroleum resins and maltenes and thus interact similarly with asphaltenes in the oil. Polyisobutylene succinic anhydrides (PIBSA's) comprising between 18 and 41 isobutylene repeat units are currently the most widely used asphaltene dispersants. Until now, however, no solvent, polymeric dispersant or combination thereof has proven to be an unqualified success at inhibiting asphaltene precipitation.

Accordingly, there is an outstanding need in the industry for improved systems and methods for inhibiting asphaltene precipitation. Desirably, such systems and methods enable reduction in the likelihood of asphaltene precipitation without undue operational cost and/or complexity.

### SUMMARY

Disclosed herein is an additive comprising an asphaltene dispersant dissolved in a solvent, wherein the asphaltene dispersant is selected from the group consisting of α-olefin/vinyl pyrrolidinone copolymers. In embodiments, the copolymer comprises at least one α-olefin selected from the group consisting of mono-α-olefins. The at least one α-olefin can be selected from the group consisting of linear α-olefins. In embodiments, the at least one α-olefin is selected from the group consisting of 1-hexadecene, 1-octadecene, 1-eicosene, 1-docosene, 1-tetracosene, 1-hexacosene, 1-octacosene and 1-triacontene. In embodiments, the at least one α-olefin is selected from the group consisting of 1-hexadecene, 1-eicosene and 1-triacontene. In embodiments, the additive comprising a copolymer of 2-pyrrolidinone, 1-ethenyl monomer. The copolymer can have an average molecular weight in the range of from about 1,000 to about 100,000, an average molecular weight in the range of from about 2,000 to about 25,000 and/or an average molecular weight in the range of from about 3,000 to about 15,000. In embodiments, the solvent is selected from the group consisting of aromatic solvents. The solvent can comprise at least one aromatic selected from the group consisting of 1-methyl naphthalene, bis-(m-phenoxyphenyl)ether, o-xylene, toluene, heavy aromatic solvents and combinations thereof. In embodiments, the solvent is selected from the group consisting of asphaltene compatible solvents.

In embodiments, the copolymer has a Hansen Solubility Parameter (HSP), δ, of greater than 16 MPa^{1/2}. In embodiments, the copolymer has an HSP of greater than 17 MPa^{1/2}. In embodiments, the copolymer has an HSP of greater than 18 MPa^{1/2}. In embodiments, the Hansen Compatibility Number of the copolymer with the solvent is less than about 6.5. In embodiments, the Hansen Compatibility Number (HCN) of the copolymer with the solvent is less than about 5.5.

In embodiments, the additive comprises a copolymer of N-vinyl-2-pyrrolidinone and at least one α-olefin selected from the group consisting of 1-hexadecene, 1-octadecene, 1-eicosene, 1-docosene, 1-tetracosene, 1-hexacosene, 1-octacosene and 1-triacontene and the solvent comprises at least one aromatic selected from the group consisting of 1-methyl naphthalene, 1,4-, bis-(m-phenoxyphenyl)ether, o-xylene, toluene and combinations thereof. In some such embodiments, the solvent comprises 1-methyl naphthalene. In some such embodiments, the HCN is less than about 4.1. In embodiments, the solvent consists essentially of 1-methylnaphthalene and the HCN is less than or about 2.7.

In embodiments, the additive comprises a copolymer of N-vinyl-2-pyrrolidinone and at least one α-olefin selected from the group consisting of 1-hexadecene, 1-octadecene, 1-eicosene, 1-docosene, 1-tetracosene, 1-hexacosene, 1-octacosene and 1-triacontene and the solvent comprises bis-(m-phenoxyphenyl)ether. In some such embodiments, the HCN between the solvent and the asphaltene aggregate is less than about 3.3. In some such embodiments, the solvent consists essentially of bis-(m-phenoxyphenyl)ether and the HCN is less than or about 3.1.

In embodiments, the additive comprises a copolymer of N-vinyl-2-pyrrolidinone and at least one α-olefin selected from the group consisting of 1-hexadecene, 1-octadecene, 1-eicosene, 1-docosene, 1-tetracosene, 1-hexacosene, 1-octacosene and 1-triacontene and the solvent comprises o-xylene. In some such embodiments, the HCN is less than about 2.3. In some such embodiments, the solvent consists essentially of o-xylene and the HCN is less than or about 5.1.

In embodiments, the additive comprises a copolymer of N-vinyl-2- pyrrolidinone and at least one α-olefin selected from the group consisting of 1-hexadecene, 1-octadecene, 1-eicosene, 1-docosene, 1-tetracosene, 1-hexacosene, 1-octacosene and 1-triacontene and the solvent comprises toluene. In some such embodiments, the HCN is less than about 2.6. In some such embodiments, the solvent consists essentially of toluene and the HCN is less than or about 6.0.

The additive can comprise from about 10 weight percent to about 90 weight percent copolymer and from about10 weight percent to about 90 weight percent solvent. In embodiments, the additive comprises from about 25 weight percent to about 75 weight percent copolymer and from about 25 weight percent to about 75 weight percent solvent. In embodiments, the additive comprises from about 40 weight percent to about 60 weight percent copolymer and from about 40 weight percent to about 60 weight percent solvent.

In embodiments, the additive comprises a copolymer of N-vinyl-2-pyrrolidinone and at least one α-olefin selected from the group consisting of 1-hexadecene, 1-octadecene, 1-eicosene, 1-docosene, 1-tetracosene, 1-hexacosene, 1-octacosene and 1-triacontene, and the molar ratio of α-olefin to vinyl pyrrolidinone is in the range of from about 0.65 to about 1.35. In embodiments, the additive comprises a copolymer of N-vinyl pyrrolidinone with at least one α-olefin selected from the group consisting of 1-hexadecene, 1-eicosene and 1-triacontene. The molar ratio of α-olefin to vinyl pyrrolidinone can be in the range of from about 0.8 to about 1.2. The molar ratio of α-olefin to vinyl pyrrolidinone can be in the range of from about 0.95 to about 1.05.

Also disclosed herein is a method of inhibiting asphaltene precipitation in a fluid, the method comprising: introducing into the fluid an asphaltene dispersant selected from the group consisting of α-olefin/vinyl pyrrolidinone copolymers. In embodiments, the fluid comprises an oil phase. In embodiments, the fluid comprises crude oil. In embodiments, the fluid is at least partially located in an oil well, an oil reservoir, crude oil distribution apparatus, crude oil refining apparatus, or a combination thereof. In embodiments, the fluid comprises an asphaltene-containing oil. In embodiments, the fluid comprises, in thermodynamic equilibrium, asphaltenes, maltenes and/or resins, and oil, and introduction of the asphaltene dispersant into the fluid maintains the thermodynamic equilibrium. In embodiments, the copolymer comprises at least one α-olefin selected from the group consisting of linear mono-α-olefins. The at least one α-olefin can be selected from the group consisting of 1-hexadecene, 1-octadecene, 1-eicosene, 1-docosene, 1-tetracosene, 1-hexacosene, 1-octacosene and 1-triacontene. The at least one α-olefin can be selected from the group consisting of 1-hexadecene, 1-eicosene and 1-triacontene. In embodiments, the asphaltene dispersant comprises a copolymer of 1-vinyl-2-pyrrolidinone monomer. In embodiments, the copolymer has an average molecular weight in the range of from about 1,000 to about 100,000. In embodiments of the method, the asphaltene dispersant is introduced as an additive comprising the asphaltene dispersant dissolved in a solvent. The solvent can be selected from the group consisting of aromatic solvents. In embodiments, the solvent comprises at least one aromatic compound selected from the group consisting of 1-methyl naphthalene, bis-(m-phenoxyphenyl)ether, o-xylene, toluene, heavy aromatic naphtha and combinations thereof.

In embodiments of the method, the fluid comprises one or more asphaltenes, and the solvent is selected from the group consisting of asphaltene compatible solvents having a HSP not different by more than about 20% from the HSP of at least one asphaltene in the fluid. In embodiments, the fluid comprises one or more asphaltenes, and the solvent is selected from the group consisting of asphaltene compatible solvents having a HSP not different by more than about 10% from the HSP of at least one asphaltene in the fluid. In embodiments, the Hansen Compatibility Number (HCN) of the copolymer with the solvent is less than about 6.5 and, in embodiments, can be less than about 5.5.

In embodiments of the method, the additive comprises from about 10 weight percent to about 90 weight percent asphaltene dispersant and from about 10 weight percent to about 90 weight percent solvent.

In embodiments, the asphaltene dispersant comprises a copolymer of N-vinyl-2-pyrrolidinone and at least one α-olefin selected from the group consisting of 1-hexadecene, 1-octadecene, 1-eicosene, 1-docosene, 1-tetracosene, 1-hexacosene, 1-octacosene and 1-triacontene and wherein the solvent comprises at least one aromatic compound selected from the group consisting of 1-methyl naphthalene, bis-(m-phenoxyphenyl)ether, o-xylene, toluene and combinations thereof.

In embodiments, the dispersant comprises a copolymer of N-vinyl-2-pyrrolidinone and at least one α-olefin selected from the group consisting of 1-hexadecene, 1-octadecene, 1-eicosene, 1-docosene, 1-tetracosene, 1-hexacosene, 1-octacosene and 1-triacontene, and the molar ratio of α-olefin to vinyl pyrrolidinone is in the range of from about 0.65 to about 1.35. In some such embodiments, the asphaltene dispersant comprises a copolymer of N-vinyl pyrrolidinone with at least one α-olefin selected from the group consisting of 1-hexadecene, 1-eicosene and 1-triacontene.

In embodiments of the method, the copolymer has a Hansen Solubility Parameter (HSP), δ, of greater than 17.5, greater than 17.75 and/or greater than 18.

The foregoing has outlined rather broadly the features and technical advantages of the invention in order that the detailed description of the invention that follows may be better understood. Additional objects, embodiments, features and advantages of the invention will be will be apparent from the following detailed description of the invention and the appended claims. It should be appreciated by those skilled in the art that the conception and the specific embodiments disclosed may be readily utilized as a basis for modifying or designing other structures for carrying out the same purposes of the invention. It should also be realized by those skilled in the art that such equivalent constructions do not depart from the spirit and scope of the invention as set forth in the appended claims.

### BRIEF DESCRIPTION OF THE DRAWING

For a detailed description of the preferred embodiments of the invention, reference will now be made to the accompanying drawing wherein:

FIG. 1 is a block diagram of a method of inhibiting asphaltene precipitation according to an embodiment of this disclosure.

### NOTATION AND NOMENCLATURE

Certain terms are used throughout the following description and claim to refer to particular system components. This document does not intend to distinguish between components that differ in name but not function.

The term ‘asphaltene’ is used herein to the n-pentane (C₅H₁₂), n-hexane (C₆H₁₄) or n-heptane (C₇H₁₆)-insoluble, toluene (C₆H₅CH₃)-soluble component(s) of carbonaceous materials, such as, but not limited to, crude oil, bitumen and coal; said component consisting primarily of carbon, hydrogen, nitrogen, oxygen, and sulfur, as well as trace amounts of other components, including, but not limited to, vanadium and nickel.

As used herein, the term ‘α-olefin’ refers to unsaturated chemical compounds containing a carbon-to-carbon double bond at the primary or alpha position, i.e. alkenes containing a carbon-to-carbon double bond at the primary or alpha position.

As used herein, the terms ‘Linear Alpha Olefins’, ‘LAO,’ Normal Alpha Olefins' and ‘NAO’ are used to refer to α-olefins having the chemical formula CₙH₂ₙ, distinguished from other mono-olefins with a similar molecular formula by linearity of the hydrocarbon chain and the position of the double bond at the primary or alpha position.

As used herein, the terms ‘Branched Alpha Olefins’ and ‘BAO’ are used to refer to α-olefins having a non-linear hydrocarbon chain.

The use of the terms ‘inhibition,’ ‘inhibiting,’ and ‘inhibit,’ when used to refer to the precipitation of asphaltenes refers to minimization of the degree of precipitation thereof. As used herein, inhibition of asphaltene precipitation includes prevention of all or some degree of precipitation.

Use of the term ‘comprising’ herein should also be understood to cover embodiments of ‘consisting of’ and ‘consisting essentially of.’

### DETAILED DESCRIPTION

Overview. Herein disclosed are systems and methods for inhibiting asphaltene precipitation. In an embodiment, the system and method incorporate an asphaltene dispersant comprising, consisting of or consisting essentially of an α-olefin/vinyl pyrrolidinone copolymer.

Chemical Additive. Herein disclosed is an additive comprising an (i.e. at least one) asphaltene dispersant which is optionally dissolved in a solvent, wherein the asphaltene dispersant is selected from the group consisting of α-olefin/vinyl pyrrolidinone copolymers.

The α-olefins are unsaturated chemical compounds containing a carbon-to-carbon double bond at the primary or alpha position, i.e. alkenes containing a carbon-to-carbon double bond at the primary or alpha position. Linear Alpha Olefins (LAO) or Normal Alpha Olefins (NAO) are olefins or alkenes with a chemical formula CₙH₂ₙ, distinguished from other mono-olefins with a similar molecular formula by linearity of the hydrocarbon chain and the position of the double bond at the primary or alpha position. The simplest acyclic alkenes, with only one double bond and no other functional groups, form a homologous series of hydrocarbons with the general formula CₙH₂ₙ. In embodiments, the asphaltene dispersant comprises a copolymer of at least one α-olefin selected from the group consisting of linear and branched mono-α-olefins. In embodiments, the at least one α-olefin is selected from the group consisting of linear α-olefins. In embodiments, the at least one α-olefin is selected from the group consisting of linear α-olefins.

In embodiments, the asphaltene dispersant comprises a copolymer of at least one α-olefin selected from the group consisting of linear α-olefins having the formula CₙH₂ₙ. In embodiments, the at least one linear α-olefin having the formula CₙH₂ₙ comprises at least 16 carbons. In embodiments, the at least one linear α-olefin comprises from about 16 to about 30 carbon atoms. In embodiments, the at least one linear α-olefin is selected from the group consisting of 1-hexadecene, 1-octadecene, 1-eicosene, 1-docosene, 1-tetracosene, 1-hexacosene, 1-octacosene and 1-triacontene. In embodiments, the at least one linear α-olefin is selected from the group consisting of 1-eicosene and 1-triacontene.

In embodiments, the additive comprises a copolymer of 1-vinyl-2-pyrrolidinone monomer, also sometimes referred to as N-vinylpyrrolidinone, NVP, N-vinyl-2-pyrrolidinone, N-vinyl-2-pyrrolidone, vinylbutyrolactam, N-vinyl-2-vinylpyrrolidone, and 2-pyrrolidinone, 1-ethenyl.

In embodiments, the α-olefin/vinyl pyrrolidinone copolymer has an average molecular weight in the range of from about 1,000 to about 100,000, from about 2,000 to about 25,000 or from about 3,000 to about 15,000.

When present, the solvent is an asphaltene compatible solvent, compatible with the asphaltenes for which the additive is to prevent precipitation and/or with the asphaltene dispersant(s). Compatibility of the solvent for use in the additive may be determined by determining the Hansen Solubility Parameter (HSP) of the solvent and/or the Hansen Compatibility Number (HCN) of the solvent with the asphaltene(s) and/or with the α-olefin/vinyl pyrrolidinone copolymer(s), as will be further discussed vide infra. As further discussed hereinbelow, in embodiments, the HSPs of the solvent and the asphaltene(s) to be inhibited from precipitating are similar; the HSPs of the solvent and the asphaltene dispersant(s) to be dissolved therein are similar; the HCN of any solvent with the asphaltene(s) is low; the HCN of any solvent with the α-olefin/vinyl pyrrolidinone copolymer(s) is low; or a combination thereof.

To date, a definitive molecular description of the building blocks of asphaltene aggregate(s) has not been cemented. Debate regarding the fundamental asphaltene molecular architecture continues. As mentioned hereinabove, according to this disclosure, asphaltenes are the n-pentane (C₅H₁₂), n-hexane (C₆H₁₄) or n-heptane (C₇H₁₆)-insoluble, toluene (C₆H₅CH₃)-soluble component(s) of carbonaceous materials, such as, but not limited to, crude oil, bitumen and coal; and consisting primarily of carbon, hydrogen, nitrogen, oxygen, and sulfur, as well as trace amounts of other components, including, but not limited to, vanadium and nickel. The molar H:C molar ratio of asphaltene(s) depends on the asphaltene source. In embodiments, the disclosed asphaltene dispersant, additive and/or method are utilized to inhibit precipitation of asphaltene(s) having a molar H:C molar ratio that is approximately 1:1.25. In embodiments, the asphaltenes have a distribution of molecular masses in the range of from about 1000 unified atomic mass units (u or Daltons, Da) to about 6000 u. In embodiments, the predominant molecular mass is approximately 2700 u.

In embodiments, the asphaltenes have an average MW in the range of from about 2500-4000 u, as measured by the ebullioscopy method. In embodiments, the asphaltenes have an average MW in the range of from about 600-6000 u, as measured by the cryoscopy method. In embodiments, the asphaltenes have an average MW in the range of from about 900-2000 u as measured via viscosity determinations. In embodiments, the asphaltenes have an average MW in the range of from about 1000-5000 u, as measured by VPO.

As mentioned hereinabove, interactions within asphaltenic aggregates are largely due to van der Waals (dispersion) forces and include π-π overlap between the aromatic-rich regions of the asphaltenes with possible enhancement of association by coulombic interactions (hydrogen-bonding) among polar oxygenic and nitrogenous moieties. In embodiments, an asphaltene dispersant is utilized to inhibit precipitation of an asphaltene aggregate fitting the ‘archipelago’ model, i.e. containing condensed aromatic-like ‘islands’ interconnected with aliphatic chains. An effective asphaltene dispersant may, thus, according to this disclosure, have a similar structure.

Noting again that liquids having similar solubility parameters, δ, will be miscible and, since polymers will dissolve in solvents/liquids whose solubility parameters are not too different from their own, the additive of this disclosure can comprise an asphaltene dispersant and a solvent, having similar Hansen Solubility Parameters (HSPs). HSPs may be determined as disclosed in Hansen, C. M., Hansen Solubility Parameters: A User's Handbook, 2007, 2^{nd} ed., CRC Press, Boca Raton, Fla. Furthermore, in embodiments, the asphaltene dispersant and the solvent used to deliver the dispersant have similar thermodynamic properties to those of the asphaltene aggregate. For example, in embodiments, the HSP of the solvent is similar to the HSP of the asphaltene dispersant, the HSP of the asphaltene dispersant is similar to the HSP of the asphaltene, or both the HSPs of the asphaltene dispersant and the HSP of any solvent used to carry the asphaltene dispersant are similar to the HSP of the asphaltene(s) to be inhibited from precipitating. In embodiments, the HSPs of two fluids are considered to be similar if they are within at least 50, 60, 70, 80, 90 or 95 percent of one another.

The basis of the HSP is the total energy of vaporization of a liquid consisting of several individual parts. These individual parts arise from (atomic) dispersion forces, (molecular) permanent dipole-permanent dipole forces and (molecular) hydrogen bonding (electron exchange). These parts may be expressed according to Eq. (1):

δ²=δ_{d}²+δₚ²+δₕ², (1)

wherein δ is the Hansen Solubility Parameter (HSP), δ_{d} is the dispersion contribution, δₚ is the polarity contribution and δₕ is the hydrogen bonding contribution. Therefore, the Hansen Solubility Parameter or HSP, δ, can be obtained as the square root of the sum of squares of the individual dispersion, polarity and hydrogen bonding contributions, as expressed in Eq. (2):

δ=√(δ_{d}²+δₚ²+δₕ²). (2)

The dispersion parameter, δ_{d}, can be calculated according to the procedures outlined by Blanks, R. F. and Prausnitz, J. M., in Ind. Eng. Chem. Fundamentals, 1964, 3(1), 1-8, which is hereby incorporated herein in its entirety for all purposes not contrary to this disclosure. According to Blanks and Prausnitz, the square root of the dispersion cohesive energy density, CED, can be used to determine the partial dispersion parameter, δ_{d}, compared to the molar volume of the liquid sample (cm³/mol) and a reduced temperature, Tᵣ. The reduced temperature can be obtained as provided in Eq. (3),

Tᵣ=T/T_{c} (3)

wherein T=298.15K and T_{c} is the critical temperature and can be estimated from Lyderson group contributions. Normal boiling point (T_{b}) can also be used to determine the partial dispersion parameter, δ_{d}, wherein:

T_{b}/T_{c}=0.567+ΣΔT−(ΣΔT)² (4)

In Eq. (4), T_{c} the critical temperature can be calculated by equation 4 or can be found in Hansen, C. M., Paint Testing Manual, No. 17, 1995, 383-404, ed. Koleske, J. V., American Society for Testing and Materials, Philadelphia, Pa.; T_{b} is the normal boiling point of the various hydrocarbon(s) and ΔT is taken as 298.15° K. It has been found that a correction factor is required for atoms much larger than carbon, including chlorine, sulfur and bromine. This correction factor is approximately 1650 kJ/mol and can be utilized to balance the solubility parameter equation, Eq. (1). Since the dispersion parameter is based on atomic forces, wherein the size of the atom/molecule is important, the aforementioned corrections are applied by first finding the dispersion cohesive energy for the atom/molecule having only C atoms. This can be determined by multiplication by the molar volume (Mᵥ) and using data published in Hansen's Solubility Parameter text previously mentioned herein above. To the mathematic solution is then added the correction factor. Dividing the sum by the molar volume (Mᵥ) and then determining the square root gives the large atom corrected (LAC) dispersion solubility parameter.

The polarity parameter, δₚ, was first assigned by Blanks and Prausnitz and required the molar volume, the dipole moment (DM), the refractive index and the dielectric constant. As such data is not readily available for most compounds; a simpler equation was developed by Hansen and Beerbower, Solubility Parameters, Kirk-Othmer Encyclopedia of Chemical Technology, Suppl. Vol., 2^{nd} ed., Standen, A., ed., Interscience, New York, 1971, pp. 889-910.

According to Hansen and Beerbower,

δₚ=37.4 (DM)N^{1/2}, (5)

wherein DM is the dipole moment in Debyes and V is the molar volume in cm³/mol. The constant 37.4 provides the δₚ parameter in SI units, MPa^{1/2}.

Finally, the hydrogen bonding contribution, δₕ, is almost always found by subtracting the polar and dispersion energies of vaporization from the total energy of vaporization. Group contribution techniques are considered reasonably reliable for most of the required calculations. Therefore, in the absence of reliable latent heat and dipole moment data, group contributions may be utilized according to the work of Small, Hoy and others.

In embodiments, the copolymer of the additive has a Hansen Solubility Parameter (HSP), δ, of greater than or equal to about 16 to about 20 MPa^{1/2}. Some of the older literature uses the Hildebrand (H) unit which in cgs units equals (cal/cm³)^{1/2}. In the later literature MPa^{1/2} is employed which is 2.0455 times larger than the Hildebrand unit.

The Hansen Compatibility Number or HCN can be used to determine compatibility of fluids. For example, according to this disclosure, the HCN can be used to determine the compatibility of a solvent and an asphaltene dispersant and the compatibility of a solvent and asphaltene dispersant with the asphaltene(s) to be inhibited from precipitating. The HCN between a first fluid and a second fluid can be calculated according to Eq. (6),

HCN=√(ΔDispersion)²+(ΔPolarity)²+(ΔHydrogen Bonding),² (6)

wherein ΔDispersion is the difference between the dispersion contributions, δ_{d}'s, of the first and second fluids, ΔPolarity is the difference between the polarity contributions, δₚ's, of the first and second fluids and ΔHydrogen Bonding is the difference between the hydrogen bonding contributions, δₕ's, of the first and second fluids.

In embodiments, an asphaltene inhibiting additive comprises at least one α-olefin/vinyl pyrrolidinone copolymer dissolved in a solvent. In embodiments, the Hansen Compatibility Number, HCN, of the copolymer and the solvent employed to make the additive is greater than or equal to 0 and less than or equal to about 6.5. In embodiments, the Hansen Compatibility Number, HCN, of the α-olefin/vinyl pyrrolidinone copolymer with the solvent of the additive is less than about 10, 9, 8, 7, 6.5, 6, 5.5, 5 or 4.5. In embodiments, the solvent comprises at least one component selected from the group consisting of 1-methyl naphthalene, 1, 4-dioxane, bis-(m-phenoxyphenyl)ether, o-xylene, 2-pinene, toluene, and combinations thereof. In embodiments, the asphaltene-inhibiting additive comprises an aromatic solvent. In embodiments, the aromatic solvent comprises at least one aromatic selected from the group consisting of 1-methyl naphthalene, bis-(m-phenoxyphenyl)ether, o-xylene, toluene, heavy aromatic naphtha and combinations and hydrocarbon mixtures thereof.

In embodiments, the asphaltene-inhibiting additive comprises from about 10 weight percent to about 90 weight percent copolymer and from about 10 weight percent to about 90 weight percent solvent from about 25 weight percent to about 75 weight percent copolymer and from about 25 weight percent to about 75 weight percent solvent, or from about 40 weight percent to about 60 weight percent copolymer and from about 40 weight percent to about 60 weight percent solvent. In embodiments, the solvent is the major component of the chemical additive (i.e. of the herein disclosed asphaltene-inhibiting additive).

In embodiments, the asphaltene-inhibiting additive comprises a copolymer of N-vinyl-2-pyrrolidinone and at least one α-olefin selected from the group consisting of 1-hexadecene, 1-octadecene, 1-eicosene, 1-docosene, 1-tetracosene, 1-hexacosene, 1-octacosene and 1-triacontene, and the molar ratio of α-olefin to vinyl pyrrolidinone is in the range of from about 0.65 to about 1.35; from about 0.8 to about 1.2; or from about 0.95 to about 1.05.

In embodiments, the additive comprises a copolymer of N-vinyl pyrrolidinone with at least one α-olefin selected from the group consisting of 1-hexadecene, 1-eicosene and 1-triacontene.

In embodiments, the additive comprises a copolymer of N-vinyl-2-pyrrolidinone and at least one α-olefin selected from the group consisting of 1-hexadecene, 1-octadecene, 1-eicosene, 1-docosene, 1-tetracosene, 1-hexacosene, 1-octacosene and 1-triacontene and the solvent comprises at least one aromatic component selected from the group consisting of 1-methyl naphthalene, bis-(m-phenoxyphenyl)ether, o-xylene, toluene, heavy aromatic naphtha and combinations thereof.

In embodiments, the solvent comprises 1-methyl naphthalene (δ=21.145). In embodiments, the solvent comprises 1-methyl naphthalene and the HCN of the solvent and the α-olefin/vinyl pyrrolidinone copolymer(s) is less than about 4.1. In embodiments, the solvent consists essentially of 1-methyl naphthalene and the HCN of the solvent and the asphaltene is less than or equal to about 2.7.

In embodiments, the solvent comprises bis-(m-phenoxyphenyl)ether. In embodiments, the solvent comprises bis-(m-phenoxyphenyl)ether (δ=20.49) and the HCN of the solvent and the α-olefin/vinyl pyrrolidinone copolymer(s) is less than about 3.3. In embodiments, the solvent consists essentially of bis-(m-phenoxyphenyl)ether and the HCN of the solvent and the asphaltene is less than or equal to about 3.1.

In embodiments, the solvent comprises o-xylene. In embodiments, the solvent comprises o-xylene and the HCN of the solvent and the α-olefin/vinyl pyrrolidinone copolymer(s) is less than about 2.3. In embodiments, the solvent consists essentially of o-xylene and the HCN of the solvent and the asphaltene is less than or equal to about 5.1.

In embodiments, the solvent comprises toluene. In embodiments, the solvent comprises toluene and the HCN of the solvent and the α-olefin/vinyl pyrrolidinone copolymer(s) is less than about 2.6. In embodiments, the solvent consists essentially of toluene and the HCN of the solvent and the asphaltene is less than or about 6.0.

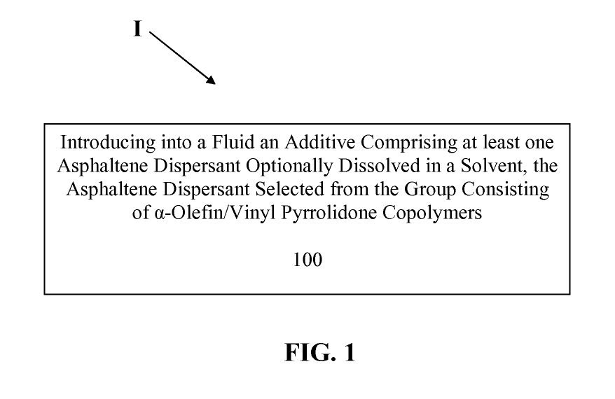

Method of Inhibiting Asphaltene Precipitation. Also disclosed herein is a method of inhibiting asphaltene precipitation in a fluid. Description of the method will be made with reference to FIG. 1, which is a block diagram of a method I of inhibiting asphaltene precipitation according to an embodiment of this disclosure.

Method I comprises introducing into a fluid an additive comprising, consisting of, or consisting essentially of at least one asphaltene dispersant selected from the group consisting of α-olefin/vinyl pyrrolidinone copolymers, and optionally dissolved in a solvent. In embodiments, the method comprises introducing into the fluid an asphaltene dispersant selected from the group consisting of α-olefin/vinyl pyrrolidinone copolymers.

The fluid to be treated for inhibition of asphaltene precipitation can comprise an oil phase. In embodiments, the fluid comprises crude oil. In embodiments, the fluid is at least partially located within an oil well, an oil reservoir, at least one component of a crude oil distribution apparatus, such as a pipeline, at least one component of a crude oil refining apparatus (such as, by way of non-limiting example, a crude unit preheat train, a crude unit side-stream pump around(s), and/or a crude unit feed to either the vacuum tower or a catalytic cracking unit) or some combination thereof. In embodiments, the fluid comprises an asphaltene-containing oil. The fluid can comprise, in thermodynamic equilibrium, asphaltenes, maltenes and/or resins, and oil, and introduction of the asphaltene dispersant (the additive) into the fluid can result in maintenance of the thermodynamic equilibrium, thus preventing asphaltene precipitation, or nearly maintaining thermodynamic equilibrium, such that precipitation is at least inhibited.

In embodiments, the α-olefin/vinyl pyrrolidinone copolymer has a Hansen Solubility Parameter (HSP), δ, of greater than or equal to about 16, 17, 18, 19 or 20 or in the range of from about 16 to about 20 MPa^{1/2}. In embodiments, the α-olefin/vinyl pyrrolidinone copolymer has a Hansen Solubility Parameter (HSP), δ, in the range of from about 15 to about 23 MPa^{1/2}. In embodiments, the α-olefin/vinyl pyrrolidinone copolymer has a Hansen Solubility Parameter (HSP), δ, in the range of from about 17.8 to about 26 MPa^{1/2}. In general, the solubility behavior of asphaltenes may be similar to that of bitumens and/or crude oils.

In embodiments, the α-olefin/vinyl pyrrolidinone copolymer is formed from at least one α-olefin selected from the group consisting of α-olefins having the formula CₙH₂ₙ. In embodiments, the α-olefin/vinyl pyrrolidinone copolymer is formed from at least one α-olefin selected from the group consisting of linear mono-α-olefins. In embodiments of the method, the at least one α-olefin is selected from the group consisting of 1-hexadecene, 1-octadecene, 1-eicosene, 1-docosene, 1-tetracosene, 1-hexacosene, 1-octacosene and 1-triacontene. In embodiments, the at least one α-olefin is selected from the group consisting of 1-eicosene and 1-triacontene. The α-olefin/vinyl pyrrolidinone copolymer may be formed from 1-vinyl-2-pyrrolidinone monomer. The α-olefin/vinyl pyrrolidinone copolymer can have an average molecular weight in the range of from about 1,000 to about 100,000 from about 2,000 to about 25,000, or from about 3,000 to about 15,000.

The asphaltene dispersant(s) can be introduced independently or as an additive comprising at least one asphaltene dispersant dissolved in a solvent. As discussed hereinabove, the solvent can be selected from the group consisting of solvents having a Hansen Compatibility Number of less than about 6.5. In embodiments, the solvent comprises at least one compound selected from the group consisting of 1-methyl naphthalene, bis-(m-phenoxyphenyl)ether, o-xylene, toluene, and combinations thereof. In embodiments, the fluid comprises one or more asphaltenes and the solvent is selected from the group consisting of asphaltene compatible solvents having a HSP not different by more than about 20% from the HSP of at least one asphaltene in the fluid.

In embodiments, the Hansen Compatibility Number (HCN) of the α-olefin/vinyl pyrrolidinone copolymer with the solvent is less than about 6.5. In embodiments, the HCN of the α-olefin/vinyl pyrrolidinone copolymer with the solvent is greater than or equal to 0 and less than or equal to about 5.5.

In embodiments, the method comprises introducing the asphaltene dispersant into the fluid as an additive solution comprising from about 10 weight percent to about 90 weight percent of one or more α-olefin/vinyl pyrrolidone copolymer(s) and from about 10 weight percent to about 90 weight percent solvent, from about 25 weight percent to about 75 weight percent copolymer and from about 25 weight percent to about 75 weight percent solvent or from about 40 to about 60 weight percent of one or more α-olefin/vinyl pyrrolidone copolymer(s) and from about 40 weight percent to about 60 weight percent solvent.

In embodiments, the dispersant comprises a copolymer of N-vinyl-2-pyrrolidinone and at least one α-olefin selected from the group consisting of 1-hexadecene, 1-octadecene, 1-eicosene, 1-docosene, 1-tetracosene, 1-hexacosene, 1-octacosene and 1-triacontene, wherein the molar ratio of α-olefin to vinyl pyrrolidinone is in the range of from about 0.65 to about 1.35, from about 0.8 to about 1.2 or from about 0.95 to about 1.05.

The asphaltene dispersant introduced into the fluid via the method can comprise a copolymer of N-vinyl pyrrolidinone with at least one α-olefin selected from the group consisting of 1-hexadecene, 1-eicosene and 1-triacontene.

In embodiments of the method, the asphaltene dispersant comprises a copolymer of N-vinyl-2-pyrrolidinone and at least one α- from the group consisting of 1-hexadecene, 1-octadecene, 1-eicosene, 1-docosene, 1-tetracosene, 1-hexacosene, 1-octacosene and 1-triacontene, carried in a solvent comprising at least one component selected from the group consisting of 1-methyl naphthalene, bis-(m-phenoxyphenyl)ether, o-xylene, toluene and combinations thereof.

In embodiments, the solvent comprises 1-methyl naphthalene. In embodiments, the solvent comprises 1-methyl naphthalene and the HCN of the solvent with the α-olefin/vinyl pyrrolidinone copolymer(s) is less than about 4.1. In embodiments, the solvent consists essentially of 1-methyl naphthalene and the HCN of the solvent and the asphaltene is less than or equal to about 2.7.

In embodiments, the solvent comprises bis-(m-phenoxyphenyl)ether. In embodiments, the solvent comprises bis-(m-phenoxyphenyl)ether and the HCN of the solvent with the α-olefin/vinyl pyrrolidinone copolymer(s) is less than about 3.3. In embodiments, the solvent consists essentially of bis-(m-phenoxyphenyl)ether and the HCN of the solvent and the asphaltene is less than or equal to about 3.1.

In embodiments, the solvent comprises o-xylene. In embodiments, the solvent comprises o-xylene and the HCN of the solvent with the α-olefin/vinyl pyrrolidinone copolymer(s) is less than about 2.3. In embodiments, the solvent consists essentially of o-xylene and the HCN of the solvent and the asphaltene is less than or equal to about 5.1.

In embodiments, the solvent comprises toluene. In embodiments, the solvent comprises toluene and the HCN of the solvent with the α-olefin/vinyl pyrrolidinone copolymer(s) is less than about 2.6. In embodiments, the solvent consists essentially of toluene and the HCN of the solvent and the asphaltene is less than or equal to about 6.0.

In embodiments, the method comprises introducing an effective amount of asphaltene-inhibiting additive into a fluid comprising asphaltenes. The additive may be added to the fluid in an amount of less than, equal to or greater than about 1,000 ppm to about 10,000 ppm vol/vol, about 100 to about 1,000 vol/vol or about 10 to about 100 ppm vol/vol.

### EXAMPLES

The following examples are presented to further illustrate the present invention and are not to be construed as unduly limiting the scope of this invention.

Hansen Solubility Parameters (HSPs) were employed to characterize two asphaltenes, three PIBSAs, several solvents which are considered good asphaltene solvents and three experimental polymers, all of which proved to be an exceptional asphaltene dispersants.

Polymers V-216, V-220 and WP-660 available from International Specialty Products, Performance and Industrial Chemicals Division, Wayne, N.J., are a C₁₆/NVP copolymer, C₂₀/NVP copolymer and C₃₀/NVP copolymer, respectivcely and received in a 95-100% active form.

Solubility Parameters of Asphaltenes and Solvents. Calculations were run using Molecular Modeling Pro (ChemSW, version 6.0.6) to determine the HSP of the asphaltenes Laquinillas Asphaltene and Athabasca Asphaltene. The dispersion parameter, δ_{d}, the polarity parameter, δₚ, and the hydrogen bonding parameter, δₕ, were determined by employing either the Hansen method (calculated by an unpublished proprietary algorithm contained in the aforementioned program), the van Krevelen and Hofzyger method (van Krevenlen, D. W., Properties of Polymers,1990, Elsevier, N.Y., p. 200-225), the Hoy method (Hoy, K. L., J. Paint Techn.,1970, 42(76)), or the Miller variation of the Small method (Miller, R. F., Evaluation of Ethylene Copolymers as Pour Depressants, in Macromolecular Solutions, 1982, Seymour, R. B. and Stahl, G. A., eds. Pergamon Press, New York, p. 84-89). The HSP was calculated from δ_{d}, δₚ and δₕ according to Eq. (2) hereinabove and the results are presented in Table 1.

TABLE 1
Asphaltene Solubility Parameter Data

| | Laquinillas | Athabasca |
|---|---|---|
| Parameter | Asphaltene (n = 1) | Asphaltene |
| Dispersion, δ_{d} (cal/cm)^{1/2} (× | 20.6441 | 20.6231 |
| 2.0455 = MPa^{1/2}) | | |
| Polarity, δₚ (MPa^{1/2}, see Eq. 5 | 1.2470 | 0.6905 |
| hereinabove) | | |
| Hydrogen Bonding, δₕ (MPa^{1/2}) | 7.323 | 6.5393 |
| Hansen Solubility Parameter, δ, | 21.94 | 21.65 |
| (MPA^{1/2}) | | |

Guinier analyses makes no assumptions about an asphaltenic aggregates geometric shape and is used simply to obtain the radius of gyration, R_{G}, which is a measure of mass distribution about the center of the mass of the asphaltene aggregate according to Eq. (7):

(R_{G})²=[(L_{cyl})²/3]+[(R_{cycl})²*[[(z+5)]/[2*(z+1)²]], (7)

wherein L_{cyl} is the average cylinder length, R_{cyl} is the average cylinder radius and z is the radial polydispersity index defined as z=(R_{cyl}/σ_{R})²−1. Asphaltenes recovered from a “whole” Hondo crude oil and upon forming aggregates in toluene gave a R_{G}=56 Å. The aggregate volume, or V_{agg}, was 128 nm³ (Verruto, V. J. and Kilpatrick, P. K., Energy and Fuels, 2007, 21(3), 1343-1349). In attempting to determine the best solvent or solvent mixtures for asphaltenes, the data determined/calculated yielded a method by which R_{G} and V_{agg} provide criterion to rank solvent/co-solvent quality and asphaltene solvency.

Solvents like decalin (also sometimes known as decahydronaphthalene and bicyclo-[4.4.0] decane), toluene, and 1-methyl naphthalene were evaluated to determine the HSP, RG and V_{Agg} values thereof. HSP was determined by one of the methods aforementioned above whereas R_{G} and V_{agg} were determined by Verruto and Kilpatrick. The results are provided in Table 2.

TABLE 2
Solvent Solubility Data and Asphaltene Aggregate Size

| | | | 1-Methyl |
|---|---|---|---|
| Parameter | Decalin | Toluene | Naphthalene |
| Dispersion, δ_{d} (cal/cm)^{1/2} × | 18.4 | 18.0 | 20.6 |
| 2.0455 = MPa^{1/2} | | | |
| Polarity, δₚ (MPa^{1/2}, see | 0 | 1.4 | 0.8 |
| Eq. 5 hereinabove) | | | |
| Hydrogen Bonding, δₕ | 0 | 2.0 | 4.7 |
| (Mpa^{1/2}) | | | |
| Hansen Solubility | 18.4 | 18.165 | 21.145 |
| Parameter, δ, (MPa^{1/2}) | | | |
| Radius of Gyration, R_{G,} | 72 | 56 | 47 |
| (Å) | | | |
| Aggregate Volume, V_{agg,} | 267 | 128 | 65 |
| (nm³) | | | |

Hexane and heptane are known to be asphaltene solubility antagonists which tend to cause precipitation of asphaltenes from crude oil, referred to by Verruto as ‘antisolvents’. The data presented in Table 2 suggests that the polynuclear aromatic moieties in/on the asphaltenes and either hexane or heptane solvent produces a thermodynamically unstable solution. Therefore, to determine which solvent or co-solvent system is the best for asphaltenes, one should not only consider the HSP but also which system's aggregates have the smallest <R_{G}> and <V_{agg}>. As seen, asphaltene solvency decreases in the order of 1-methylnaphthalene>toluene>decalin>n-heptane or n-hexane. However, if the H/C ratio or ‘aromaticity index’ of the solvent is considered, it is observed that, as the solvents become increasingly aromatic with decreasing H/C, they also become increasingly better solvents for asphaltenes.

Asphaltene Dispersancv Tests with Various Dispersants. The effectiveness of various dispersants was studied by determining the amount of sediment produced, relative to a blank, as a function of the amount of dispersant added to a sample. Three modified-PIBSAs, LUBRIZOL 5948 available from Lubrizol Corporation, Wickliffe, Ohio, KEMELIX 7475X available from Croda Chemicals, New Castle, Del. and FLOWSOLVE 111 available from J. D. Horizons, Cheshire, U.K, were studied, as well as Ganex V-216, a 2-pyrrolidinone, 1-ethenyl-, hexadecyl copolymer (C₁₆H₃₂.C₆H₉NO)ₓ, Ganex V-220, a 2-pyrrolidinone, 1-ethenyl-, copolymer with eicosene, (C₂₀H₄₀.C₆H₉NO)ₓ and Ganex WP-660, a 2-pyrrolidinone, 1-ethenyl-, copolymer with 1-triacontene, (C₃₀H₆₀.C₆H₉NO)ₓ.

For the control (blanks), 12.5 mL of hexane was added to each of four (4) 15 mL centrifuge tubes. The hexane was dosed with a desired amount of crude/refined oil stock solution. Typically, the following dosages were chosen: 50, 100, 200 and 300 μL. An Eppendorf or Drummond pipette was utilized to accurately measure the oil. The mixture was shaken vigorously for 60 seconds. The tubes were allowed to stand for one hour, after which time the percent sedimentation due to gravity was observed and recorded. It is noted that, as the amount of oil in the hexane increased, the amount of precipitate also increased and the supernatant solution became lighter.

The Asphaltene Dispersant Test (ADT) procedure comprised adding a desired amount of dispersant to each tube. Typically, the following dosages were chosen 0 (blank), 10, 50, 100 and 200 ppm. For some crude, i.e. California, up to 1000 ppm of dispersant or more may be required in laboratory field studies to reflect conditions in the field. It is noted that 1.25% v/v solutions of dispersant in toluene may be prepared so that dosing is facilitated, i.e. 10 μL added, equals 10 ppm. Next, 12.5 mL of hexane was added and the combination mixed until the dispersant solution was uniformly incorporated into the hexane solution. Subsequently, sufficient crude oil or asphaltene solution was added to the hexane so that the blank gave a statistically repeatable percentage (%) of sediment. The tubes were shaken vigorously for 60 seconds and then allowed to stand. After 60 minutes, the percent sediment due to gravity was recorded.

The percent dispersion was calculated from the volume of sediment in the treated samples (Sₜᵣₑₐₜ) and in the blank (S_{blank}) according to Eq. (8):

% Dispersed=[(S_{blank}−Sₜᵣₑₐₜ)/S_{blank}]×100. (8)

Sedimentation test results are presented in Table 3.

TABLE 3
Sedimentation Test Results

| | Dispersant | Sediment | Amount |
|---|---|---|---|
| Sample | Dosage (ppm) | Volume (mL) | Dispersed (%) |
| Blank(s) | 0 | 1.5, 1.45, 1.3, 1.4 | 0 |
| Lubrizol 5948 | 250 | 1.1 | 21.99 |
| | 500 | 0.85 | 39.71 |
| | 750 | 0.7 | 50.35 |
| | 1000 | 0.5 | 64.54 |
| Kemelix 7475X | 250 | 1.2 | 14.89 |
| | 500 | 0.9 | 36.17 |
| | 750 | 0.9 | 36.17 |
| | 1000 | 0.8 | 43.26 |
| FlowSolve 111 | 250 | 0.8 | 43.26 |
| | 500 | 0.7 | 50.36 |
| | 750 | 0.6 | 57.45 |
| | 1000 | 0.4 | 71.63 |
| Ganex WP-660 | 250 | 0 | 100.0 |
| (C₃₀H₆₀•C₆H₉NO)ₓ | 500 | 0 | 100.0 |
| | 750 | 0 | 100.0 |
| | 1000 | 0 | 100.0 |
| Ganex V-220 | 250 | 0 | 100.0 |
| (C₂₀H₄₀•C₆H₉NO)ₓ | 500 | 0 | 100.0 |
| | 750 | 0 | 100.0 |
| | 1000 | 0 | 100.0 |
| Ganex V-216 | 250 | 0 | 100.0 |
| (C₁₆H₃₂•C₆H₉NO)ₓ | 500 | 0 | 100.0 |
| | 750 | 0 | 100.0 |
| | 1000 | 0 | 100.0 |

From the results in Table 3, it is apparent that the dispersancy of the asphaltene is improved via utilization of the non-PIBSA dispersants. Ganex V-216, V-220 and WP-660, all three of which are α-olefin/2-pyrrolidinone copolymers outperformed the three PIBSA-based chemicals significantly.

Solubility Data for PIBSAs. The HSPs of two PIBSAs and three α-olefin/2-pyrrolidinone copolymers were determined by calculating δ_{d}, δₚ and δₕ according to the procedures discussed hereinabove. The HSPs were then calculated from δ_{d}, δₚ and δₕ according to Eq. (2) hereinabove. The results are presented in Table 4 hereinbelow.

As shown in Table 4, a PIBSA containing n=18 isobutylene groups per succinic anhydride group had a calculated solubility parameter of 16.51. The second PIBSA, with n=41 isobutylene groups per succinic anhydride group, had a lower HSP than the PIBSA with n=18 isobutylene groups, exhibiting a δ of 16.221. The lower HSP for the PIBSA with n=41 was expected because of the difference observed between the molecules δₚ and the larger difference between the molecules δₕ which was at least 30%.

It is noted that PIBSAs with 18 and 41 isobutylene groups per succinic anhydride group were tested because they were the only PIBSA isomers having a TSCA and a CAS registry number.

TABLE 4
Solubility Data

| | Dispersion, | | | |
|---|---|---|---|---|
| | δ_{d} | | | |
| | (cal/cm)^{1/2} × | Polarity, δₚ | Hydrogen | |
| | 2.0455 = | (MPa^{1/2}; | Bonding, δₕ | HSP, δ |
| Sample | MPa^{1/2}) | see Eq. 5) | (MPa^{1/2}) | (MPa^{1/2}) |
| PIBSA (n = 18) | 16.0301 | 1.0222 | 3.8403 | 16.51 |
| PIBSA (n = 41) | 15.9937 | 0.4633 | 2.5853 | 16.221 |
| WP-660 | 17.4549 | 1.9875 | 2.8889 | 17.804 |
| V-220 | 17.47 | 2.73 | 3.38 | 18.00 |
| V-216 | 17.48 | 3.21 | 3.67 | 18.147 |

For the monomer of Ganex WP-660, (C₃₀H₆₀.C₆H₉NO)₁, δ was calculated to be 17.804, which is higher than the HSPs calculated for either PIBSA. The Hansen Solubility Parameter, δ, was also calculated for a monomer of the C₂₀ copolymer (V-220) (C₂₀H₄₀.C₆H₉NO)₁ and was found to be 18.00. Finally, for the monomer of Ganex V-216, (C₁₆H₃₂.C₆H₉NO)₁, δ was calculated to be 18.147.

To complete yet another analysis of the physical chemical data for the various asphaltenes and asphaltene dispersants, the solubility parameter distance, Rₐ, according to Eq. (9) herein below was applied. Rₐ is the modified distance between the Hansen Solubility Parameter (HSP) for a solvent (1) and polymer (2). The constant “4” has been empirically useful to convert spheroidal plots of solubility into spherical ones using δ_{D} and either of the other two parameters. This equation has been used with success in well over 1000 HSP correlations with a computer program that optimizes a solubility sphere according to equation 8 where all good solvents are within the sphere and the bad ones are outliers. Theoretically, less effective interactions between an asphaltene (2) and a dispersant under investigation (1) would then be predicted by increasing radius of the sphere.

(Rₐ)²=4(δ_{D2}=δ_{D1})²+(δ_{P2}−δ_{P1})²+(δ_{H2}−δ_{H1}). (9)

Rₐ has the same units as solubility parameter.

TABLE 5
Hansen Solubility Parameter Distances, Rₐ

| Asphaltene | Dispersant | Rₐ |
|---|---|---|
| Laquinillas Asphaltene (n = 1) | PIBSA (n = 18) | 9.86 |
| | PIBSA (n = 41) | 10.46 |
| | WP-660 | 7.80 |
| | V-220 | 7.62 |
| | V-216 | 7.56 |
| Athabasca Asphaltene | PIBSA (n = 18) | 9.58 |
| | PIBSA (n = 41) | 10.07 |
| | WP-660 | 7.41 |
| | V-220 | 7.34 |
| | V-216 | 7.35 |

As indicated in Table 5, results from both calculations and physical testing suggests that, for the Laquinillas Asphaltenes, V-216 appears to be more effective as a dispersant thereof than is WP-660, which is more effective as a dispersant of Laquinillas Asphaltene than a PIBSA with n=18, which is more effective as a dispersant than a PIBSA with n=41 based upon calculated Rₐ. values. Similarly, for Athabasca Asphaltenes, VS-220 appears to be as effective a dispersant as is WP-660, which is more effective as a dispersant of Athabasca Asphaltene than a PIBSA with n=18, which is more effective as a dispersant than a PIBSA with n=41.

While preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, and so forth). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, and the like.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the preferred embodiments of the present invention. The disclosures of all patents, patent applications, and publications cited herein are hereby incorporated by reference, to the extent they provide exemplary, procedural or other details supplementary to those set forth herein.

What is claimed is:

1. An additive comprising an asphaltene dispersant dissolved in a solvent, wherein the asphaltene dispersant is selected from the group consisting of α-olefin/vinyl pyrrolidinone copolymers.

2. The additive of claim 1 wherein the copolymer comprises at least one α-olefin selected from the group consisting of mono-α-olefins.

3. The additive of claim 2 wherein the at least one α-olefin is selected from the group consisting of linear α-olefins.

4. The additive of claim 3 wherein the at least one α-olefin is selected from the group consisting of 1-hexadecene, 1-octadecene, 1-eicosene, 1-docosene, 1-tetracosene, 1-hexacosene, 1-octacosene and 1-triacontene.

5. The additive of claim 4 wherein the at least one α-olefin is selected from the group consisting of 1-hexadecene, 1-eicosene and 1-triacontene.

6. The additive of claim 1 comprising a copolymer of 2-pyrrolidinone, 1-ethenyl monomer.

7. The additive of claim 1 wherein the copolymer has an average molecular weight in the range of from about 1,000 to about 100,000.

8. The additive of claim 7 wherein the copolymer has an average molecular weight in the range of from about 2,000 to about 25,000.

9. The additive of claim 8 wherein the copolymer has an average molecular weight in the range of from about 3,000 to about 15,000.

10. The additive of claim 1 wherein the solvent is selected from the group consisting of aromatic solvents.

11. The additive of claim 10 wherein the solvent comprises at least one aromatic selected from the group consisting of 1-methyl naphthalene, bis-(m-phenoxyphenyl)ether, o-xylene, toluene, heavy aromatic solvents and combinations thereof.

12. The additive of claim 1 wherein the solvent is selected from the group consisting of asphaltene compatible solvents.

13. The additive of claim 12 wherein the copolymer has a Hansen Solubility Parameter (HSP), δ, of greater than 16 MPa^{1/2}.

14. The additive of claim 13 wherein the copolymer has an HSP of greater than 17 MPa^{1/2}.

15. The additive of claim 14 wherein the copolymer has an HSP of greater than 18 MPa^{1/2}.

16. The additive of claim 1 wherein the Hansen Compatibility Number of the copolymer with the solvent is less than about 6.5.

17. The additive of claim 16 wherein the Hansen Compatibility Number (HCN) of the copolymer with the solvent is less than about 5.5.

18. The additive of claim 17 comprising a copolymer of N-vinyl-2-pyrrolidinone and at least one α-olefin selected from the group consisting of 1-hexadecene, 1-octadecene, 1-eicosene, 1-docosene, 1-tetracosene, 1-hexacosene, 1-octacosene and 1-triacontene and wherein the solvent comprises at least one aromatic selected from the group consisting of 1-methyl naphthalene, 1,4-, bis-(m-phenoxyphenyl)ether, o-xylene, toluene and combinations thereof.

19. The additive of claim 18 wherein the solvent comprises 1-methyl naphthalene.

20. The additive of claim 19 wherein the HCN is less than about 4.1.

21. The additive of claim 19 wherein the solvent consists essentially of 1-methylnaphthalene and wherein the HCN is less than or about 2.7.

22. The additive of claim 18 wherein the solvent comprises bis-(m-phenoxyphenyl)ether.

23. The additive of claim 22 wherein the HCN between the solvent and the asphaltene aggregate is less than about 3.3.

24. The additive of claim 22 wherein the solvent consists essentially of bis-(m-phenoxyphenyl)ether and wherein the HCN is less than or about 3.1.

25. The additive of claim 18 wherein the solvent comprises o-xylene.

26. The additive of claim 25 wherein the HCN is less than about 2.3.

27. The additive of claim 25 wherein the solvent consists essentially of o-xylene and wherein the HCN is less than or about 5.1.

28. The additive of claim 18 wherein the solvent comprises toluene.

29. The additive of claim 28 wherein the HCN is less than about 2.6.

30. The additive of claim 28 wherein the solvent consists essentially of toluene and wherein the HCN is less than or about 6.0.

31. The additive of claim 1 comprising from about 10 weight percent to about 90 weight percent copolymer and from about10 weight percent to about 90 weight percent solvent.

32. The additive of claim 1 comprising from about 25 weight percent to about 75 weight percent copolymer and from about 25 weight percent to about 75 weight percent solvent.

33. The additive of claim 1 comprising from about 40 weight percent to about 60 weight percent copolymer and from about 40 weight percent to about 60 weight percent solvent.

34. The additive of claim 1 comprising a copolymer of N-vinyl-2-pyrrolidinone and at least one α-olefin selected from the group consisting of 1-hexadecene, 1-octadecene, 1-eicosene, 1-docosene, 1-tetracosene, 1-hexacosene, 1-octacosene and 1-triacontene, wherein the molar ratio of α-olefin to vinyl pyrrolidinone is in the range of from about 0.65 to about 1.35.

35. The additive of claim 34 comprising a copolymer of N-vinyl pyrrolidinone with at least one α-olefin selected from the group consisting of 1-hexadecene, 1-eicosene and 1-triacontene.

36. The additive of claim 34 wherein the molar ratio of α-olefin to vinyl pyrrolidinone is in the range of from about 0.8 to about 1.2.

37. The additive of claim 36 wherein the molar ratio of α-olefin to vinyl pyrrolidinone is in the range of from about 0.95 to about 1.05.

38. A method of inhibiting asphaltene precipitation in a fluid, the method comprising:
introducing into the fluid an asphaltene dispersant selected from the group consisting of α-olefin/vinyl pyrrolidinone copolymers.

39. The method of claim 38 wherein the fluid comprises an oil phase.

40. The method of claim 38 wherein the fluid comprises crude oil.

41. The method of claim 40 wherein the fluid is at least partially located in an oil well, an oil reservoir, crude oil distribution apparatus, crude oil refining apparatus or some combination thereof.

42. The method of claim 38 wherein the fluid comprises an asphaltene-containing oil.

43. The method of claim 38 wherein the fluid comprises, in thermodynamic equilibrium, asphaltenes, maltenes and/or resins, and oil, and wherein introduction of the asphaltene dispersant into the fluid maintains the thermodynamic equilibrium.

44. The method of claim 38 wherein the copolymer comprises at least one α-olefin selected from the group consisting of linear mono-α-olefins.

45. The method of claim 44 wherein the at least one α-olefin is selected from the group consisting of 1-hexadecene, 1-octadecene, 1-eicosene, 1-docosene, 1-tetracosene, 1-hexacosene, 1-octacosene and 1-triacontene.

46. The method of claim 45 wherein the at least one α-olefin is selected from the group consisting of 1-hexadecene, 1-eicosene and 1-triacontene.

47. The method of claim 38 comprising a copolymer of 1-vinyl-2-pyrrolidinone monomer.

48. The method of claim 38 wherein the copolymer has an average molecular weight in the range of from about 1,000 to about 100,000.

49. The method of claim 38 wherein the asphaltene dispersant is introduced as an additive comprising the asphaltene dispersant dissolved in a solvent.

50. The method of claim 49 wherein the solvent is selected from the group consisting of aromatic solvents.

51. The method of claim 50 wherein the solvent comprises at least one aromatic compound selected from the group consisting of 1-methyl naphthalene, bis-(m-phenoxyphenyl)ether, o-xylene, toluene, heavy aromatic naphtha and combinations thereof.

52. The method of claim 51 wherein the fluid comprises one or more asphaltenes, and wherein the solvent is selected from the group consisting of asphaltene compatible solvents having a HSP not different by more than about 20% from the HSP of at least one asphaltene in the fluid.

53. The method of claim 51 wherein the fluid comprises one or more asphaltenes, and wherein the solvent is selected from the group consisting of asphaltene compatible solvents having a HSP not different by more than about 10% from the HSP of at least one asphaltene in the fluid.

54. The method of claim 49 wherein the Hansen Compatibility Number (HCN) of the copolymer with the solvent is less than about 6.5.

55. The method of claim 54 wherein the Hansen Compatibility Number (HCN) of the copolymer with the solvent is less than about 5.5.

56. The method of claim 49 wherein the additive comprises from about 10 weight percent to about 90 weight percent asphaltene dispersant and from about 10 weight percent to about 90 weight percent solvent.

57. The method of claim 49 wherein the asphaltene dispersant comprises a copolymer of N-vinyl-2-pyrrolidinone and at least one α-olefin selected from the group consisting of 1-hexadecene, 1-octadecene, 1-eicosene, 1-docosene, 1-tetracosene, 1-hexacosene, 1-octacosene and 1-triacontene and wherein the solvent comprises at least one aromatic compound selected from the group consisting of 1-methyl naphthalene, bis-(m-phenoxyphenyl)ether, o-xylene, toluene and combinations thereof.

58. The method of claim 38 wherein the dispersant comprises a copolymer of N-vinyl-2-pyrrolidinone and at least one α-olefin selected from the group consisting of 1-hexadecene, 1-octadecene, 1-eicosene, 1-docosene, 1-tetracosene, 1-hexacosene, 1-octacosene and 1-triacontene, wherein the molar ratio of α-olefin to vinyl pyrrolidinone is in the range of from about 0.65 to about 1.35.

59. The method of claim 58 wherein the asphaltene dispersant comprises a copolymer of N-vinyl pyrrolidinone with at least one α-olefin selected from the group consisting of 1-hexadecene, 1-eicosene and 1-triacontene.

60. The method of claim 38 wherein the copolymer has a Hansen Solubility Parameter (HSP), δ, of greater than 17.5

61. The method of claim 60 wherein the copolymer has an HSP of greater than 17.75.

62. The method of claim 61 wherein the copolymer has an HSP of greater than 18.
